(19) United States
(12) Patent Application Publication
(10) Pub. No.: US 2022/0331267 A1
(43) Pub. Date: Oct. 20, 2022

(54) METHODS AND COMPOSITIONS FOR TREATMENT OF DEMYELINATING DISORDERS

(71) Applicant: Rush University Medical Center, Chicago, IL (US)

(72) Inventors: Kalipada PAHAN, Skokie, IL (US); Avik ROY, Chicago, IL (US)

(73) Assignee: Rush University Medical Center, Chicago, IL (US)

(21) Appl. No.: 17/640,698

(22) PCT Filed: Sep. 2, 2020
(86) PCT No.: PCT/US2020/048991

§ 371 Date: Mar. 4, 2022

Related U.S. Application Data
(60) Provisional application No. 62/896,150, filed on Sep. 5, 2019.

(51) Int. Cl. A61K 31/05; A61K 31/015; A61K 31/21; A61K 9/70; A61K 9/16; A61P 25/00

(52) U.S. Cl. CPC A61K 31/05; A61K 31/015; A61K 31/21; A61K 9/7038; A61K 9/167; A61P 25/00

(57) ABSTRACT

Methods of modulating peroxisome proliterator-activated receptor β (PPARβ) activity in a cell in a subject in need thereof are provided. The methods include administering an effective amount of a PPAPβ ligand to the subject where the PPAPβ ligand is selected from 1,3-Di-tertbutyl benzene (DBB), 2,4-Di-tertbutyl phenol (DBP), Methyl palmitate (MePA), 2,6-Di-tertbutyl-4-Methyl Phenol (DBMP), 3,4-Di-tertbutyl-Phenol (3,4-DBP), 2,3-Di-tertbutyl-Phenol (2,3-DBP), and 2,6-Di-tertbutyl-Phenol (2,6-DBP).

### RELATED APPLICATIONS

This application claims the benefit of the filing date of U.S. Provisional Patent Application No. 62/896,150, filed Sep. 5, 2019, the contents of which are hereby incorporated by reference.

### FEDERALLY SPONSORED RESEARCH OR DEVELOPMENT

This invention was made with government support under Grant No. NS097426, awarded by the National Institutes of Health. The Government has certain rights in the invention.

### BACKGROUND

Oligodendrocytes are a type of glial cells that form myelin wraps around neuronal axons and protect nerve conduction in CNS. These cells are differentiated from oligodendroglial progenitor cells OPCs (1) and largely populated in the white-matter region of CNS such as cortex (2), corpus callosum (2) and cerebellum (3). Although, oligodendrocytes are extensively present in the cerebellum, it is not known how these oligodendrocytes are differentiated. One theory suggests that oligodendrocytes of the cerebellum are differentiated in the ventral midbrain and then migrated into the cerebellum during early embryogenesis (4). According to that theory, OPCs located at neuroepithelium of ventral rhombomere1 differentiate and then migrate to the cerebellum. Another concept supports that OPCs differentiate locally in the cerebellar ventricular zone to provide majority of oligodendrocytes in the cerebellum (5). Despite all these conflicting ideas, it is now well-accepted that OPCs are highly active in the cerebellum from early embryogenesis to postnatal period (6). In fact, these cells are capable of differentiating into oligodendrocytes even if the cerebellum of an adult brain is damaged during demyelinating diseases or injury (7). Moreover, cerebellar OPCs can be easily isolated from the postnatal brain and differentiated to oligodendrocytes in vitro with cell culture methods (8-10). Taken together, this evidence suggests that cerebellar OPCs are very resourceful tools to study oligodendroglial differentiation in the adult brain. However, the molecular mechanism involved in the differentiation of cerebellar OPCs into oligodendrocytes still remains elusive.

Peroxisome proliferator-activated receptor β or δ (PPARβ/δ) and its two isomers PPARα and γ belong to a class of nuclear hormone receptors (11) named PPARs (12,13). Although PPARs are primarily involved in energy metabolism, PPARβ has been long known to participate in a diverse range of biological functions including anti-inflammation (14,15), immuno-modulation (16), anti-oxidation (17,18) and myelinogenesis (19,20). In the central nervous system (CNS), PPARβ is primarily expressed in oligodendrocytes (21). Other glial cells including astrocytes and microglia express very low level of PPARβ (22). While studying the role of PPARβ in oligodendrocytes, our research suggests that PPARβ directly controls the transcription of different myelin genes including PLP, MBP, CNPase and MOG (19) in oligodendrocytes suggesting that PPARβ is directly involved in the differentiation of OPCs to oligodendrocytes (23) via upregulation of oligodendroglial markers. However, unlike PPARα and γ, the cellular regulation of PPARβ has not been fully explored (24). Accordingly, until now PPARβ is the only PPAR, which does not have any known cellular ligands. Although long-chain fatty acids are considered to be endogenous regulators of PPARβ, these molecules are also known to activate other PPARs (25,26) with variable efficiency. In fact, one study suggests that long-chain fatty acid metabolites display stronger affinity towards the lipophilic LBD of PPARα (27). Therefore identifying a selective physiological ligand of PPARβ has a strong biological importance. The ligand binding domain (LBD) of PPARβ is 268 amino acids long domain with a 1300 A° large, Y-shaped cavity surrounded by six alpha helices and two beta sheets (28). Because of the large size of the LBD, PPARβ is capable of holding structurally diverse group of ligands.

Here specific ligands of PPARβ are identified in mouse brain cerebellum. The identified ligands of PPARβ include 1,3-Di-tertbutyl benzene (DBB), 2,4-Di-tertbutyl phenol (DBP), Methyl palmitate (MePA). Both DBB and DBP are di tertiary butylated aromatic compounds, whereas MePA is a methyl derivative of hexadecanoic acid. For identification, we adopted an affinity purification method, in which GFP-PPARβLBD protein was incubated with cerebellar lysates and then trapped in a GFP column followed by purification and detection with Electron ionization-GCMS method. With the help of this method, we were able to isolate these three compounds in mouse brain cerebellum. Interestingly, our cheminformatic analyses coupled with protein thermal shift and FRET assay further validated that these three ligands are strong ligands of PPARβ. Moreover, our cell culture analyses demonstrated that these three agonists of PPARβ upregulated the expression of myelin genes and stimulated the differentiation of OPC into oligodendrocytes.

The DBB, DBP and MePA ligands induce the activation of PPARβ in the central nervous system. What is needed in the art are PPARβ ligands for modulating PPARβ activity and for treatment of disorders such as demyelinating disorder.

### BRIEF SUMMARY

Methods of modulating peroxisome proliferator-activated receptor β (PPARβ) activity in a cell in a subject in need thereof are provided. The methods include administering an effective amount of a PPARβ ligand to the subject, the PPARβ ligand selected from the group consisting of 1,3-Di-tertbutyl benzene (DBB), 2,4-Di-tertbutyl phenol (DBP), Methyl palmitate (MePA), 2,6-Di-tertbutyl-4-Methyl Phenol (DBMP), 3,4-Di-tertbutyl-Phenol (3,4-DBP), 2,3-Di-tertbutyl-Phenol (2,3-DBP), and 2,6-Di-tertbutyl-Phenol (2,6-DBP).

### BRIEF DESCRIPTION OF THE DRAWINGS

FIG. 1A-1T. The role of DBB, DBP and MePA in the differentiation of OPC into oligodendrocytes and myelination. (FIG. 1A) A plate-based oligonucleotide probe binding assay of PPARβ with different concentrations of DBB (1, 2 and 5 μM), DBP (1, 2 and 5 μM) and MePA (0.1, 0.2 and 0.5 μM). Briefly, mouse primary OPCs were treated with different concentrations of molecules for 1 hr, lysed and measured for DNA probe binding activity of PPARβ. Results are confirmed after three independent experiments with *p<0.05 vs. controls for DBB (p=0.023), DBP (p=0.038) and MePA (p=0.045) respectively. PPRE-luciferase activity was measured in the mouse primary OPCs after 4 hrs of stimulation with increasing concentrations of (FIG. 1B) DBB, (FIG. 1C) DBP and (FIG. 1D) MePA. ***p<0.001 vs. control for DBB (p=0.00013), DBP (p=0.00026) and MePA (p=0.00088). (FIG. 1E) Realtime and (FIG. 1F) semi-quantitative RT-PCR analyses of PLP, MOG, MBP and CNPase in OPCs treated with 1 μM of DBB, DBP and 0.5 μM of MePA. Benzene was included as negative control. **p<0.01 vs. control for PLP, MOG, MBP, and CNPase. Results are mean±SD of three independent experiments. (FIG. 1G) Immunoblot followed by (FIG. 1H) relative densitometric analyses of PLP and MBP in mouse primary OPCs treated with increasing concentrations of DBB for 18 hrs. Band intensity was measured with image J software and then normalized with respective actin band. *p<0.05 (=0.041) vs. PLP control and *p<0.05 (=0.026) vs. MBP control. Similarly, (FIG. 1I) immunoblot followed by the (FIG. 1J) relative densitometric analyses were performed for PLP and MBP in OPCs treated with different concentrations of DBP. **p<0.01 vs. control of PLP (p=0.0067) and MBP (p=0.0075). (FIG. 1K) Immunoblot and (FIG. 1L) relative densitometric analyses for PLP and MOG in OPCs treated with MePA for 18 hrs. *p<0.05 vs. control PLP (p=0.039) and **p<0.01 vs control MOG (p=0.0038). (FIG. 1M) Dual immunostaining of MBP (red) and NG2 (green) in OPCs treated with 1 μM DBB, DBP and 0.5 μM of MePA for 24 hrs. (N) Mean fluorescence intensity (MFI) of MBP was measured from total 15 cells per group with three cells from 5 different images per group. One way ANOVA was performed to test the significance of mean, which is F_{3,56}=39.45 (>F_{c}=4.80), **p<0.001 (=0.000378). (FIG. 1O) A Pearson correlation analyses of MBP and NG2 resulted negative correlation with r=−0.4023 and p<0.001. (FIG. 1P) Dual immunofluorescence analyses of PLP (red) and A2B5 (green) in OPCs treated with 1 μM DBB, DBP and 0.5 μM of MePA for 24 hrs. (FIG. 1Q) Mean fluorescence intensity (MFI) of MBP was measured from total 15 cells per group with three cells from 5 different images per group. One way ANOVA was performed to test the significance of mean, which is F_{3,56}=69.40(>F_{c}=4.80), **p<0.001 (=0.000378). (FIG. 1R) A Pearson correlation analyses of PLP and A2B5 resulted negative correlation with r=−0.4693 and p<0.001. (FIG. 1S) Mouse primary OPCs were seeded in polycaprolactone nanofiber matrix and then stimulated with DBB, DBP and MePA for 48 hrs followed by immunostaining with MBP. Phase contrast image of nanofibers were merged with fluorescence image of MBP and displayed. (FIG. 1T) Numbers of MBP-ir cells of different treatment groups were counted per sq.mm of nanofiber matrix and presented as a histogram. Five independent images per group were included for counting. One way ANOVA was adopted to test the significance of mean between groups that results F_{3,16}=21.2 (>Fc=4.80); **p<0.01 (=0.0084). ***p<0.001 (=0.00094 for DBB, 0.00086 for DBP and 0.00065 for MePA) vs. control.

FIG. 2A-2B. PPRE-driven luciferase assay in Pparb-null OPCs. PPRE luciferase activity was assayed in Pparb-null OPCs pre-infected with lentiviral particles of (FIG. 2A) GFP vector and (FIG. 2B) fIPPARβ. One way ANOVA (factor: genotype) results F_{3, 9}=12.451 (>F_{c}=4.80); **p<0.01 (=0.00325). Results are mean±SD of three independent experiments.

FIG. 3A-3J. DBB, DBP and MePA stimulated the differentiation of oligodendrocytes and myelination via PPARβ. PPRE-luciferase activity was measured in Pparb-null mouse primary OPCs infected with empty vector (blue bars), flpparβ (green bars), H412Lpparβ (red bars) and V304Mpparβ (purple bars)-constructed lentiviral particles. After 48 hrs of viral infection, these cells were transfected with PPRE-luciferase gene for another 24 hrs followed by stimulation with (FIG. 3A) DBB, (FIG. 3B) DBP and (FIG. 3C) MePA for additional 4 hrs. *p<0.05, **p<0.01 and ***p<0.001 vs. respective control. (FIG. 3D) Dual immunostaining of MBP (red) and NG2 (green) in Pparb-null OPCs treated with 1 μM DBB (FIG. 3Dii), DBP (FIG. 3Diii) and 0.5 μM of MePA (FIG. 3Div) for 24 hrs. (FIG. 3E) Mean fluorescence intensity (MFI) of MBP was measured in Pparb-null OPCs infected with lentiviral particles constructed with pparb genes followed by 24 hrs treatment with (FIG. 3Ei) DBB, (FIG. 3Eii) DBP and (FIG. 3Eiii) MePA. Total 50 cells per group had been included in counting with 10 cells from each image from 5 different images per group. One way ANOVA was performed to test the significance of mean in DBB-treated groups, which results F_{4, 186}=98.82 (>F_{c}=4.80), ***p<0.0001 (=0.0000417). Similar analysis of DBP-treated groups results F_{4, 209}=167.1 (>F_{c}=4.80), ***p<0.0001 (=0.0000076). For MePA-treated groups, one way ANOVA results F (4, 233)=196.9; ***p<0.0001 (=0.0000013). Pparb-null OPCs were seeded in polycaprolactone nanofiber matrix and then transduced with GFP (FIG. 3F), GFPflpparβ followed by stimulation with (FIG. 3G) vehicle, (FIG. 3H) 1 μM DBB, (FIG. 3I) 1 μM DBB, and (FIG. 3J) 0.5 μM MePA. After 24 hrs, cells were immunostained with MBP. Phase contrast image of nanofibers were merged with fluorescence image of MBP and displayed along with fluorescence images. Results were confirmed after three independent experiments.

### DETAILED DESCRIPTION

The embodiments disclosed below are not intended to be exhaustive or to limit the scope of the disclosure to the precise form in the following description. Rather, the embodiments are chosen and described as examples so that others skilled in the art may utilize its teachings.

Methods of modulating peroxisome proliferator-activated receptor β (PPARβ) activity in a cell in a subject are provided. In some aspects, the PPARβ is modulated in a cell in the central nervous system (CNS). In some aspects, the cell is in the brain or the spinal cord. In some aspects, the cell may be a type of glial cell. The cell may be an oligodendroglial progenitor cell. An effective amount of a PPARβ ligand may be administered to the subject. The PPARβ ligand may be an aromatic compound with two tertiary butyl groups. In some embodiments, the PPARβ ligand may selected from the group consisting of 1,3-Di-tertbutyl benzene (DBB), 2,4-Di-tertbutyl phenol (DBP) and Methyl palmitate (MePA). In some embodiments, the PPARβ ligand may selected from the group consisting of 1,3-Di-tertbutyl benzene (DBB), 2,4-Di-tertbutyl phenol (DBP), Methyl palmitate (MePA), 2,6-Di-tertbutyl-4-Methyl Phenol (DBMP), 3,4-Di-tertbutyl-Phenol (3,4-DBP), 2,3-Di-tertbutyl-Phenol (2,3-DBP) and 2,6-Di-tertbutyl-Phenol (2,6-DBP).

The PPARα ligands may be administered to treat a demyelinating disorder.

“Treating”, “treat”, or “treatment” within the context of the instant invention, means an alleviation of symptoms associated with a disorder or disease, or halt of further progression or worsening of those symptoms, or prevention or prophylaxis of the disease or disorder. For example, within the context of this invention, successful treatment may include an alleviation of symptoms related to demyelinating disorders. The treatment may include administering an effective amount of a PPARβ ligand to the subject that results in an alleviation of symptoms associated with a disorder or disease, or halt of further progression or worsening of those symptoms, or prevention or prophylaxis of the disease or disorder.

By way of non-limiting example, demyelinating disorders may be selected from multiple sclerosis (MS), X-Adrenoleukodystrophy (X-ALD), Adrenomyeloneuropathy (AMN), Neuromyelitis optica (Devic's disease), acute-disseminated encephalomyelitis (ADEM), acute haemorrhagic leucoencephalitis (AHL), progressive multifocal leukoencephalopathy (PML), Krabbe disease, HIV dementia and other demyelinating disorders.

The term “subject” or “patient” as used herein, refers to a mammal, in some aspects a human.

In some embodiments, practice of the present invention will employ, unless otherwise indicated, conventional techniques of molecular biology, immunology, microbiology, cell biology and recombinant DNA, which are within the skill of the art. See e.g., Sambrook, Fritsch and Maniatis, MOLECULAR CLONING: A LABORATORY MANUAL, (Current Edition); CURRENT PROTOCOLS IN MOLECULAR BIOLOGY (F. M. Ausubel et al. eds., (Current Edition)); the series METHODS IN ENZYMOLOGY (Academic Press, Inc.): PCR 2: A PRACTICAL APPROACH (Current Edition) ANTIBODIES, A LABORATORY MANUAL and ANIMAL CELL CULTURE (R. I. Freshney, ed. (1987)). DNA Cloning: A Practical Approach, vol. I & II (D. Glover, ed.); Oligonucleotide Synthesis (N. Gait, ed., Current Edition); Nucleic Acid Hybridization (B. Hames & S. Higgins, eds., Current Edition); Transcription and Translation (B. Hames & S. Higgins, eds., Current Edition); Fundamental Virology, 2nd Edition, vol. I & II (B. N. Fields and D. M. Knipe, eds.)

Pharmaceutical Compositions

The ligands described herein may be used alone or in compositions together with a pharmaceutically acceptable carrier or excipient. Pharmaceutical compositions of the disclosed herein may comprise a therapeutically effective amount of one or more ligands of PPARβ, including one or more of the PPARβ ligands shown in Table I, together with one or more pharmaceutically acceptable carriers.

TABLE 1
Exemplary PPARβ Ligands

| | Name | Structure |
|---|---|---|
| 1 | 1,3-Di- tertbutyl benzene (DBB) | 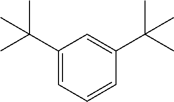 |
| 2 | 2,4-Di- tertbutyl phenol (DBP) | 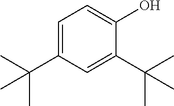 |
| 3 | Methyl palmitate (MePA) | 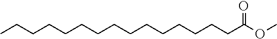 [SMILES: CCCCCCCCCCCCCCCC(=O)OC] |
| 4 | 2,6-Di- tertbutyl- 4- Methyl Phenol (DBMP) | 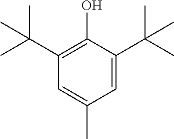 [SMILES: Cc1cc(C(C)(C)C)c(O)c(C(C)(C)C)c1] |
| 5 | 3,4-Di- tertbutyl- Phenol (3,4- DBP) | 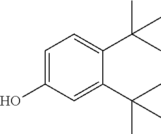 |
| 6 | 2,3-Di- tertbutyl- Phenol (2,3- DBP) | 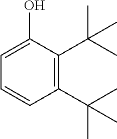 |
| 7 | 2,6-Di- tertbutyl- Phenol (2,6- DBP) | 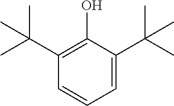 |

As used herein, the term “pharmaceutically acceptable carrier” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringers solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. Other suitable pharmaceutically acceptable excipients are described in “Remington's Pharmaceutical Sciences,” Mack Pub. Co., New Jersey, 1991, incorporated herein by reference.

The ligands described herein may be administered to humans and animals in dosage unit formulations containing conventional nontoxic pharmaceutically acceptable carriers, adjuvants, and vehicles as desired.

Methods of formulation are well known in the art and are disclosed, for example, in Remington: The Science and Practice of Pharmacy, Mack Publishing Company, Easton, Pa., 19th Edition (1995). Pharmaceutical compositions for use in the present invention can be in the form of sterile, non-pyrogenic liquid solutions or suspensions, coated capsules or lipid particles, lyophilized powders, or other forms known in the art.

Compositions of the invention may be formulated for delivery as a liquid aerosol or inhalable dry powder. Liquid aerosol formulations may be nebulized predominantly into particle sizes that can be delivered to the terminal and respiratory bronchioles.

Liquid dosage forms for oral administration include pharmaceutically acceptable emulsions, microemulsions, solutions, suspensions, syrups and elixirs. In addition to the active ligands, the liquid dosage forms may contain inert diluents commonly used in the art such as, for example, water or other solvents, solubilizing agents and emulsifiers such as ethyl alcohol, isopropyl alcohol, ethyl carbonate, EtOAc, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Solid dosage forms for oral administration include capsules, tablets, pills, powders, and granules. In such solid dosage forms, the active ligand is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, acetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

The active ligands can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active ligand may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a ligand of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulations, ear drops, and the like are also contemplated as being within the scope of this invention.

The ointments, pastes, creams and gels may contain, in addition to an active ligand of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Ligands of the invention may also be formulated for use as topical powders and sprays that can contain, in addition to the ligands of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches may also be used for providing controlled delivery of a ligand to the body. Such dosage forms can be made by dissolving or dispensing the ligand in the proper medium. Absorption enhancers can also be used to increase the flux of the ligand across the skin. The rate can be controlled by either providing a rate controlling membrane or by dispersing the ligand in a polymer matrix or gel. The ligands of the present invention can also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition to a ligand of the present invention, stabilizers, preservatives, excipients, and the like. The preferred lipids are the phospholipids and phosphatidyl cholines (lecithins), both natural and synthetic. Methods to form liposomes are known in the art. See, for example, Prescott (ed.), “Methods in Cell Biology,” Volume XIV, Academic Press, New York, 1976, p. 33 et seq.

Aerosolized formulations of the compositions described herein may be delivered using an aerosol forming device, such as a jet, vibrating porous plate or ultrasonic nebulizer, preferably selected to allow the formation of an aerosol particles having with a mass medium average diameter predominantly between 1 to 5 μm. Further, the formulation preferably has balanced osmolarity ionic strength and chloride concentration, and the smallest aerosolizable volume able to deliver effective dose of the ligands of the invention to the site of the infection. Additionally, the aerosolized formulation preferably does not impair negatively the functionality of the airways and does not cause undesirable side effects.

Aerosolization devices suitable for administration of aerosol formulations of the invention include, for example, jet, vibrating porous plate, ultrasonic nebulizers and energized dry powder inhalers, that are able to nebulize the formulation of the invention into aerosol particle size predominantly in the size range from 1-5 μm. Predominantly in this application means that at least 70% but preferably more than 90% of all generated aerosol particles are within 1-5 μm range. A jet nebulizer works by air pressure to break a liquid solution into aerosol droplets. Vibrating porous plate nebulizers work by using a sonic vacuum produced by a rapidly vibrating porous plate to extrude a solvent droplet through a porous plate. An ultrasonic nebulizer works by a piezoelectric crystal that shears a liquid into small aerosol droplets. A variety of suitable devices are available, including, for example, AERONEB and AERODOSE vibrating porous plate nebulizers (AeroGen, Inc., Sunnyvale, Calif.), SIDESTREAM nebulizers (Medic-Aid Ltd., West Sussex, England), PARI LC and PARI LC STAR jet nebulizers (Pari Respiratory Equipment, Inc., Richmond, Va.), and AEROSONIC (DeVilbiss Medizinische Produkte (Deutschland) GmbH, Heiden, Germany) and ULTRAAIRE (Omron Healthcare, Inc., Vernon Hills, Ill.) ultrasonic nebulizers.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-propanediol or 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form may be accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations may also be prepared by entrapping the drug in liposomes or microemulsions, which are compatible with body tissues.

A ligand described herein can be administered alone or in combination with other ligands, for a possible combination therapy being staggered or given independently of one another. Long-term therapy is equally possible as is adjuvant therapy in the context of other treatment strategies, as described above. Other possible treatments are therapy to maintain the patient's status after the initial treatment, or even preventive therapy, for example in patients at risk.

Effective amounts of the ligands of the invention generally include any amount sufficient to detectably an inhibition or alleviation of symptoms. The amount of active ingredient that may be combined with the carrier materials to produce a single dosage form will vary depending upon the host treated and the particular mode of administration. It will be understood, however, that the specific dose level for any particular patient will depend upon a variety of factors including the activity of the specific ligand employed, the age, body weight, general health, sex, diet, time of administration, route of administration, rate of excretion, drug combination, and the severity of the particular disease undergoing therapy. The therapeutically effective amount for a given situation can be readily determined by routine experimentation and is within the skill and judgment of the ordinary clinician.

If the ligand is administered in combination with another compound, the term “amount that is effective to modulate PPARβ activity” is understood to mean that amount of a ligand in combination with the additional compound to achieve the desired effect. In other words, a suitable combination therapy according to the current invention encompasses an amount of the ligand and an amount of the additional compound, either of which when given alone at that particular dose would not constitute an effective amount, but administered in combination would be an “amount that is effective to modulate PPARβ activity”.

It will be understood, however, that the total daily usage of the ligands and compositions of the present invention will be decided by the attending physician within the scope of sound medical judgment. The specific therapeutically effective dose level for any particular patient will depend upon a variety of factors including the disorder being treated and the severity of the disorder; the activity of the specific ligand employed; the specific composition employed; the age, body weight, general health, sex and diet of the patient; the time of administration, route of administration, and rate of excretion of the specific ligand employed; the duration of the treatment; drugs used in combination or coincidental with the specific ligand employed; and like factors well known in the medical arts.

The dose of a ligand to be administered to warm-blooded animals, for example humans of approximately 70 kg body weight, is preferably from approximately 3 mg to approximately 5 g, more preferably from approximately 10 mg to approximately 1.5 g, most preferably from about 100 mg to about 1000 mg per person per day, divided preferably into 1 to 3 single doses which may, for example, be of the same size. Usually, children receive half of the adult dose.

Results

Identification of unique cerebellar ligands of PPARβ: PPARβ regulates the expression of myelin genes in vitro in cultured oligodendrocytes (19). However, it is not known if PPARβ regulates the expression of these genes in cerebellum, one of the heavily myelinated regions of the brain. To test the expression of myelin proteins, first we performed dual immunofluorescence analyses of myelin marker MBP together with PPARβ in the cerebellar tissue of wild-type (n=3), Pparb-null (n=3) and Ppara-null (n=3) mice. Interestingly, our double immunolabeling analyses clearly indicated that both granular (GL) and molecular layers (ML) of wild-type cerebellum strongly expressed MBP, whereas the expression of that protein had been strongly attenuated in the cerebellum of Pparb-null, but not Ppara-null mice, suggesting that the expression of myelin gene MBP is tightly regulated with PPARβ, but not PPARα. Moreover, DAB immunostaining of MBP and immunoblot analyses of other myelin genes including PLP and MOG have corroborated that PPARβ indeed is involved in the expression of myelin genes in cerebellum. While analyzing the role of PPARβ in the protection of myelin architecture, LFB staining followed by the quantitative assessment of axonal diameter and myelin thickness with high-resolution transmission electron microscopy clearly indicated that there was a significant loss of myelin around the axons of cerebellar neurons in Pparb-null mice. Taken together, these results suggest that PPARβ plays critical role not only in the expression of myelin genes, but also in preserving myelin architecture of cerebellar neurons. Therefore, regulation of PPARβ in cerebellum should be studied with prime importance. PPARβ is a nuclear receptor that requires binding with intracellular ligand to exert its biological function. Therefore, next we are interested to identify if cerebellum has any intracellular ligand for PPARβ. To test that, we performed a GFP-based affinity purification procedure (25,29) in which cerebellar lysate of wild-type cerebellum was incubated with recombinant GFP-PPARα LBD and GFP-PPARβ LBD proteins, then passed through GFP column, eluted, fractionated with chloroform-methanol procedure, and finally analyzed for small molecules in the chloroform fraction with electron ionization gas chromatography (EI-GCMS) method as described elsewhere (29). Consequently, EI-GCMS analysis of GFP-PPARβ LBD-affinity-purified cerebellar lysate detected three specific compounds with characteristic base peaks of m/z=175, 191 and 74. Searching NIST MS library further indicated that these three compounds are 1, 3-ditertiary butyl benzene (DBB), 2, 4-ditertiary butyl phenol (DBP) and methyl palmitate (MePa). Interestingly, while analyzing GFP-PPARα LBD purified fraction, we were unable to detect all three compounds suggesting that these three molecules are specific ligands for PPARβ. Relative abundances of DBB, DBP and MePA were adjusted based on the peak area of internal standard Trans-O-Dithiane-4, 5-diol and then quantified with a peak integration statistics as shown in Table 2. A part of the eluted fraction was electrophoresed and stained with Coomassie blue to nullify the loading error of GFP-PPARαLBD and GFP-PPARβ LBD used for affinity purification. Accordingly, we detected two matching bands of both GFP-PPARαLBD and GFP-PPARβLBD, both having mol. wt. of 64 kDa, negating the possibility of unequal loading of sample in MS column. Taken together, our results suggest that DBB, DBP and MePA are three specific biological ligands of PPARβ in cerebellum.

TABLE 2
Peak Integration Statistics

| | | Peak R.T, | Sample | Internal | Relative | |
|---|---|---|---|---|---|---|
| Sample Name | Ion, m/z | minutes | peak | Standard | abundance | % RA |
| DBB | 175 | | | 9500000 | 0 | 0 |
| PPARα pulled-down | | | | | | |
| DBB | 175 | 7.75 | 210000 | 590000 | 0.355932203 | 35.593 |
| PPARβ pulled-down | | | | | | |
| DBP | 191 | | | 9500000 | 0 | 0 |
| PPARα pulled-down | | | | | | |
| DBP | 191 | 9.45 | 230000 | 590000 | 0.389830508 | 38.983 |
| PPARβ pulled-down | | | | | | |
| MePA | 74 | | | 9500000 | 0 | 0 |
| PPARα pulled-down | | | | | | |
| MePA | 74 | 11.73 | 240000 | 590000 | 0.406779661 | 40.678 |
| PPARβ pulled-down | | | | | | |

Peak integration statistics of DBB, DBP, and MePA in cerebellar lysate pulled down with GFP-PPARα and PPARβLBD proteins.

Characterization of Cerebellar ligands of PPARβ: Next, we wanted to characterize the binding of these three ligands in the LBD of PPARβ. We first performed a rigid body docking analysis of DBB, DBP and MePa in the LBD of PPARβ with the help of SwissDock in silico docking tool. This online docking tool generated 100 probable complexes for each compound. Representative images displaying the most favorable energetic conformation for each ligand with surrounding amino acids within 3 A° distance were identified. Two tertiary butyl sidechains of DBB and DBP are located very close to the S3 beta-sheet of PPARβ, which contains two critical hydrophobic amino acids L302 and V304. These two amino acid residues together with V297, another nonpolar amino acid located at adjacent a helix, might engage Van der Waals (VW) interactions with the tertiary butyl sidechains of DBB and DBP. However, among all three amino acids, the aliphatic sidechain of V304 was found to be closest to these compounds and therefore considered as a primary contributor of VW force. Interestingly, a cheminformatics-based high-throughput analysis of 22 structurally relevant compounds shown in Table 3 followed by validation with protein thermal shift assay revealed that aromatic compounds with two tertiary butyl groups (n=6) might have highest affinity towards the LBD of PPARβ, whereas aromatic compounds with two isopropyl (n=4), one tertiary butyl (n=5) and one isopropyl (n=6) substituents have less affinity. Ranking of all these compounds were performed combining three selection criteria including bulkiness of the side group, free energy change (ΔG) and the extent of melting curve shift of PPARβ protein. Based on that analysis, aromatic compounds with only two tertiary butyl groups displayed strong affinity towards PPARβ, whereas aromatic molecule burdened with three or more tertiary butyl groups or having less than two or no tertiary butyl group failed to exhibit strong affinity towards PPARβ. On the other hand, MePa with fairly large size of aliphatic sidechain and a polar head displayed a dispersed docked pose in the LBD. Based on the most stable structure, we observed that negatively charged polar head of MePa formed a hydrogen bond with the H412 residue of H11α helix in one arm (H^{ε2}→O² 2.113 A°) and its long hydrophobic chain was stabilized by the three unpolar amino acids V297, L302 and V304 in the other arm of Y-shaped LBD.

TABLE 3
High-throughput screening of 22 structurally
similar phenolic compounds

| | ΔG | E_{full fitness} | ΔTm | |
|---|---|---|---|---|
| Name | (kCal/mol) | (kCal/mol) | (° C.) | Rank |
| 2,6-DB-4M Phenol* | −7.19 | −2355.817 | 17.19 | 1 |
| 2,4-DB Phenol (DBP)* | −7.13 | −2356.18 | 8.42 | 2 |
| 3,4-DB Phenol* | −7.46 | −2338.61 | 8.25 | 3 |
| 2,3-DB Phenol* | −7.17 | −2321.8 | NA | 4 |
| 2,6-DB Phenol* | −7.02 | −2356 | 6.67 | 5 |
| 1,3-DB Benzene (DBB)* | −7.25 | −2360.5 | 4.04 | 6 |
| 2.5-DI, 6-M Phenol** | −6.69 | −2371.22 | NA | 7 |
| 3,6-DI Phenol** | −6.65 | −2374.25 | NA | 8 |
| 2,4-DI Phenol** | −6.48 | −2374 | 2.32 | 9 |
| 3,4-DI Phenol** | −6.22 | −2360.48 | NA | 10 |
| 5-B-Pyrogallol*** | −6.81 | −2379.98 | | 11 |
| 2,4,6 TB Phenol*** | −6.69 | −2345.07 | 0.44 | 12 |
| 3-E, 6-B Phenol*** | −6.45 | −2366.73 | NA | 13 |
| 2-B, 6-M Phenol*** | −6.41 | −2363.37 | 0.72 | 14 |
| 2-B, 5-M Phenol*** | −6.45 | −2365.24 | 0.28 | 15 |
| 2-B, 5-M Benzene*** | −6.53 | −2366.62 | NA | 16 |
| 4 M 5 I Benzene 1,3 diol^{#} | −6.47 | −2366.62 | NA | 17 |
| 4-I, 5-M Phenol^{#} | −6.31 | −2372.65 | 0.15 | 18 |
| 3-I, 4-M Phenol^{#} | −6.26 | −2370.86 | NA | 19 |
| 2-M, 5-I Phenol^{#} | −6.13 | −2372 | NA | 20 |
| 3,4,5-TE Phenol^{#} | −6.05 | −2362.56 | NA | 21 |
| 3-E, 4-M Phenol^{#} | −5.99 | −2374.64 | NA | 22 |

The list of phenolic compounds categorized based on their affinity towards PPARβ. Three conditions including bulkiness of the side groups, free energy change (ΔG), and melting temperature shift (ΔTₘ) are applied during the categorization of these molecules. DB = Di-tertiary butyl; B = tertiary butyl; DI = Di-isopropyl; I = Isopropyl; TB = Tri-tertiary butyl; M = Methyl; E = ethyl; TE = Tri-ethyl.
*indicates compounds with maximum affinity,
**indicates compounds with moderate affinity,
***indicates compounds with low affinity and
^{#}indicates compounds with very low affinity towards PPARβ.

Next, we wanted to evaluate if these endogenous ligands served as agonists of PPARβ. To test that, first we performed a protein thermal shift assay of PPARβ with increasing doses of DBB, DBP and MePA. Interestingly, increasing doses of all three ligands significantly shifted the melting curve of PPARβ towards the positive direction of X axis to an extent of more than 2° C. suggesting all three compounds are strong agonists of PPARβ. The shift of each melting curve was calculated by subtracting the melting temperature (Tₘ) of PPARβ protein+ligand with Tₘ of PPARβ protein alone. Any shift (ΔTₘ) with more than 2° C. would be considered as significant. Accordingly, as low as 1 μM of DBB (ΔTₘ=2.14° C.), 500 nM of DBP (ΔTₘ=5.22° C.) and 100 nM of MePA (ΔTₘ=5.28° C.) incurred significant shift of PPARβ protein. Moreover, a curve-fit analysis of maximum fluorescence as a function of increasing doses of ligand further revealed that the half-maximal dose (EC₅₀) of binding was approximately 500 nM for DBB, 200 nM for DBP and 50 nM for MePA. In order to nullify the specificity of these ligands to PPARα, we performed similar thermal shift assay with PPARα protein. Surprisingly, all these compounds elicited a negligible shift (ΔTₘ<2° C.) in the melting of PPARα protein indicating that these molecules are not agonists of PPARα protein. To further confirm if these compounds are genuine agonists of PPARβ we performed time resolved FRET (TR-FRET) analysis. Interestingly, when plotted the normalized fluorescence as a function of logarithmic value of increasing doses of DBB, DBP and MePa, a sigmoidal curve was resulted for each compound with EC₅₀=525 nM for DBB, 226 nM for DBP and 72 nM for MePA. Taken together, our results have identified that DBB, DBP and MePA are three novel intracellular ligands of PPARβ in cerebellum and also established that all these compounds were strong agonists of PPARβ.

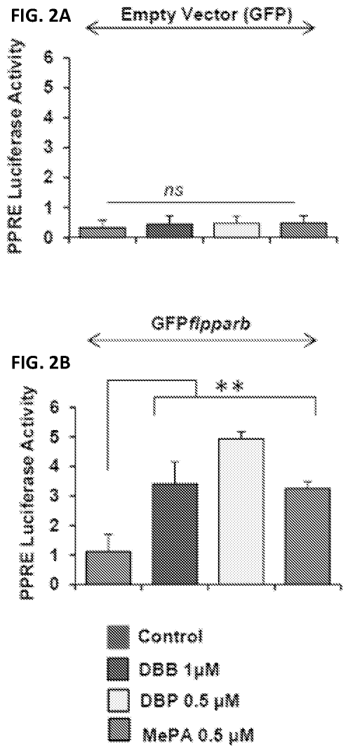

Validation of Valine 304 (V304) and Histidine 412 (H412) as two important residues of PPARβ LBD for its binding with DBB, DBP and MePA. Next, we wanted to validate the molecular interactions of these ligands with the LBD of PPARβ. Our in silico analysis already revealed that isopropyl side chain of V304 located at S3 betasheet could engage a VW interaction with all three ligands, whereas hydrogen attached to ^{ε2}N of H412 located at H11 α-helix could be involved in hydrogen boding interaction with carbonyl oxygen of MePA. To confirm the involvement of these residues, next we adopted site-directed mutagenesis strategy to mutate V304 residue to Methionine (V304M) and H412 residue to Leucine (H412L). Before performing site-directed mutation, we performed in silico docking study to confirm if V304M mutation of PPARβ indeed impaired interactions with DBB and DBP. Accordingly, the rigid-body docking analysis displayed that mutation of V304 to Methionine pushed both the molecules towards the core of the LBD causing the significant impairment of VDW interaction. On the other hand, mutation of H412 to non-ionic leucine completely attenuated H-bonding with MePA. However, V304M mutation partially augments polarity to S3 beta sheet of LBD and H412L mutation brings non-polarity to H11α helix. Therefore, there is a significant possibility of structural instability in PPARβ protein because of these mutations. To verify that, we performed protein thermal shift assay of both V304M and H412L proteins. Subsequently, we did not observe any significant change in Tₘ in both V304M (ΔTₘ=0.33° C.<2° C.) and H412L (ΔTₘ=1.77° C.<2° C.) mutants of PPARβ when compared with the Tₘ of non-mutated fIPPARβ (FIG. 2D-F) alone (Tₘ=51.98° C.) suggesting that the overall conformation of both V304M and H412L mutants of PPARβ was very similar to the wild type PPARβ. Interestingly, according to our thermal shift assay, V304M PPARβ significantly impaired the binding of DBP (ΔTₘ=0.35° C.<2° C.) and MePa (ΔTₘ=1.37° C.<2° C.) but not DBB (ΔTₘ=2.80° C.>2° C.). On the other hand, DBB, DBP and MePA strongly shifted the melting curve of H412L at a degree of 5.95° C., 8.17° C. and 4.03° C. respectively suggesting that H412L mutation was not only unable to nullify the binding of DBB, DBP and MePA in the LBD of PPARβ, but displayed enhanced interactions with all three ligands suggesting that H412L mutant could be a ligand-active mutant of PPARβ.

So far the interactions of these cerebellar ligands with PPARβ had been validated in cell free system. Therefore, next, we wanted to confirm these interactions in vitro in a cell-bound system. To test that, Pparb-null mouse primary OPCs were isolated from P1 murine neonates, infected with viral particles of GFP, GFPflpparb, GFPV304Mpparb, GFPH412Lpparb constructs for 48 hrs. After that, OPCs were homogenized, passed through the GFP-affinity column, eluted, fractionated with chloroform-methanol, and finally analyzed with GCMS to quantify these ligands. Accordingly, our GCMS analyses successfully detected both DBB and DBP in affinity-purified nuclear extract of GFPflpparb-, but not GFP-, transduced Pparb-null OPCs suggesting that both DBB and DBP have selective affinity towards PPARβ. Interestingly, V304M mutation significantly inhibited the binding of DBP, but did not impact the binding of DBB as evident in the chromatogram of affinity-purified lysate of GFPV304Mpparb infected cells. On the other hand, H412L mutation not only restored the binding of DBP, but significantly increased the binding of DBB with PPARβ as detected in affinity-purified fraction of GFPH412Lpparb-infected cells suggesting that H412L mutant is indeed a ligand-active mutant of PPARβ. While evaluating the interaction of PPARβ with MePA, chromatogram of GFPV304Mpparb-infected OPCs displayed significantly low binding of MePA with PPARβ, however, the binding of MePA was not altered in GFPH412Lpparb-transduced Pparb-null OPCs when compared with GFPflpparb-infected cells suggesting that H412L mutation did not impair the binding of MePA with PPARβ. For quantification of peak, we performed a dilution series with diluting each chloroform-extracted sample by 80, 160, 500 and 1000 times with chloroform and then performed GCMS analysis to see if peak intensities of DBB, DBP and MEPA followed a liner pattern. Consistently, we observed that H412L mutant displayed significantly high affinity with DBB, whereas V304M mutation completely nullified the interaction of PPARβ with DBP and MePA and partly with DBB. Throughout these analyses, we used dodecane as an internal standard. The result was summarized with peak integration statistics shown in Table 4, which was performed to quantify the relative abundances of these ligands. Collectively, our data suggest that V304 and H412 residues of PPARβ are critical for binding with endogenous regulators.

TABLE 4
Peak integration statistics

| | Ion, | Peak R.T, | Peak Area | Peak Area IC | Adjusted | |
|---|---|---|---|---|---|---|
| Sample Name | m/z | Minutes | (counts*min) | (counts*min) | Peak Area | RA |
| DBB | 175 | 4.448 | 2432735.657 | 59736211.14 | 0.040724639 | 1 |
| GFPflpparβ | | | | | | |
| DBB | 175 | 4.452 | 1154227.391 | 7373153.318 | 0.292171788 | 3.843977776 |
| GFPV304Mpparβ | | | | | | |
| DBB | 175 | 4.458 | 39594685.88 | 62363623.04 | 0.634900347 | 15.59007918 |
| GFPH412Lpparβ | | | | | | |
| DBP | 191 | 5.582 | 35812770.53 | 59736211.14 | 0.599515266 | 1 |
| GFPflpparβ | | | | | | |
| DBP | 191 | 5.582 | 1012730.266 | 7373153.318 | 0.137353571 | 0.229108013 |
| GFPV304Mpparβ | | | | | | |
| DBP | 191 | 5.59 | 46332980.29 | 62363623.04 | 0.742948822 | 1.239249214 |
| GFPH412Lpparβ | | | | | | |
| MePA | 74 | 7.083 | 645178.2997 | 59736211.14 | 0.010800456 | 1 |
| GFPflpparβ | | | | | | |
| MePA | 74 | 7.083 | 8717.1793 | 7373153.318 | 0.001182286 | 0.109466346 |
| GFPV304Mpparβ | | | | | | |
| MePA | 74 | 7.058 | 588149.9318 | 62363623.04 | 0.009430978 | 0.873201798 |
| GFPH412Lpparβ | | | | | | |

Peak integration statistics of GCMS analyses adopted to detect DBB, DBP and MePA in Pparb-null OPCs after transduction with different pparβ plasmids. Adjusted area was derived after dividing peak area of ligand peaks with the peak of dodecane.

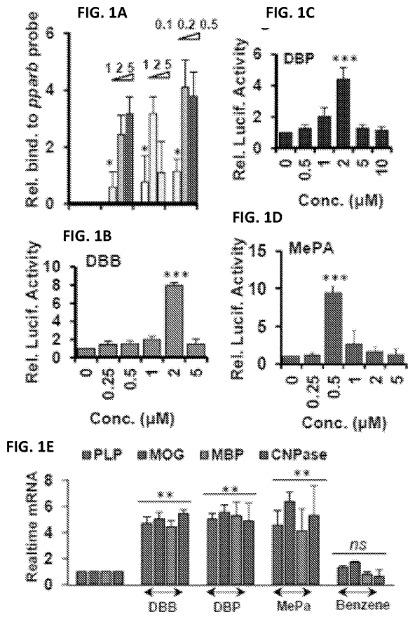

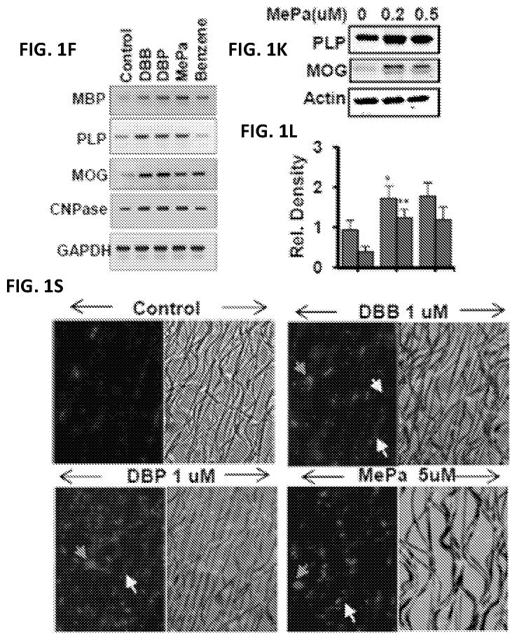

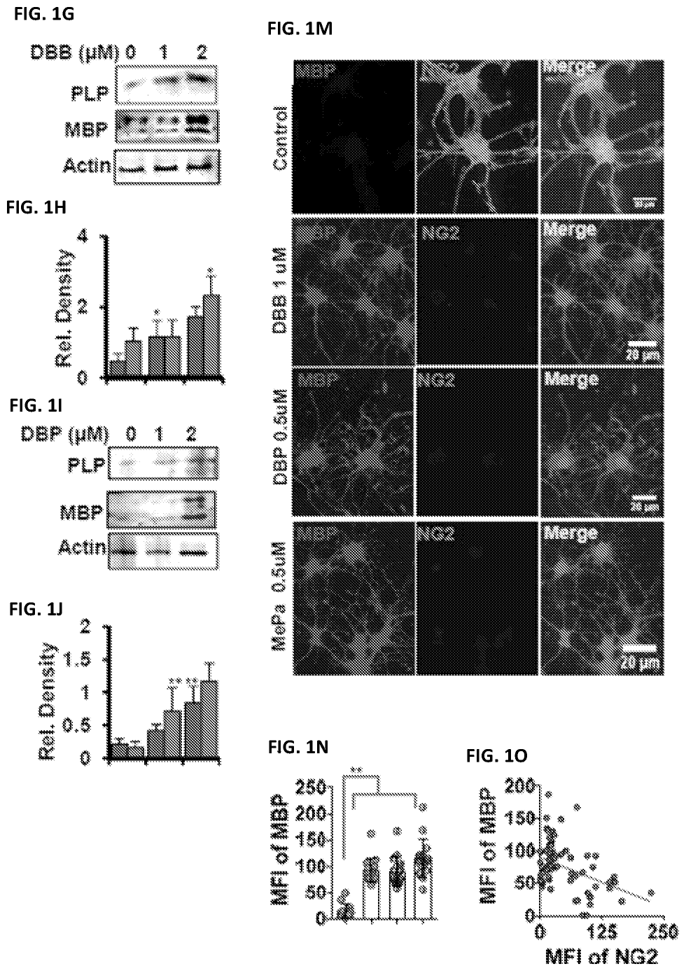

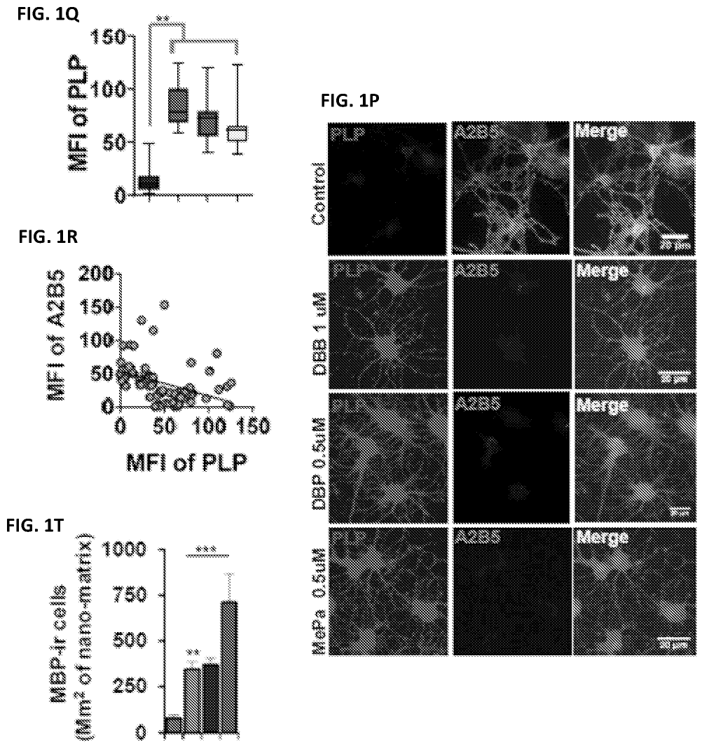

DBB, DBP and MePA stimulate the differentiation of OPC to oligodendrocytes and promote myelination. Since PPARβ regulates the expression of myelin genes, next we wanted to study if these molecules could upregulate the expression of myelin genes in cultured OPCs. First, we performed a plate-based PPRE probe binding assay to measure the DNA binding activity of PPARβ in response to increasing doses of DBB (green), DBP (blue) and MePA (red) (FIG. 1A). Briefly, OPCs were treated with different doses of ligands for 1 hr, lysed, and nuclear extracts were prepared and then added in a plate pre-coated with PPRE probe. Interestingly, increasing doses of all three ligands stimulated PPRE probe binding activity suggesting that all three ligands induced the transcriptional activity of PPARβ. To further confirm, we performed PPRE-driven luciferase assay in cultured OPCs treated with different concentrations of DBB (FIG. 1B), DBP (FIG. 1C), and MePA (FIG. 1D). Briefly, OPCs were transfected with PPRE-luciferase for 48 hrs and then treated with these compounds for additional 5 hrs. After that, cells were lysed and assessed for PPRE-luciferase activity. Interestingly, we observed that all three ligands increased the PPRE-luciferase activity in a dose-dependent manner suggesting that DBB, DBP and MePA are potent transcriptional inducers of PPARβ. Next, we monitored the expression of downstream myelin genes of PPARβ in cultured OPCs. Consistently, all these ligands stimulated the mRNA expression of different PPARβ-controlled genes including PLP, MOG, MBP and CNPase in OPCs as we confirmed with both real time (FIG. 1E) and semi-quantitative RTPCR (FIG. 1F) analyses. Similar to mRNA expression, immunoblot analyses also revealed that these ligands stimulated the expression of myelin proteins including PLP, MBP and MOG (FIG. 1G-L) in cultured OPCs. These results suggest that all three intracellular regulators could upregulate the expression of myelin genes via PPARβ. However, it is not known if these compounds stimulate the differentiation of OPCs to oligodendrocytes. To test that, we performed a double immunofluorescence analysis of OPC marker NG2 and oligodendroglial marker MBP in cultured OPCs treated with cerebellar ligands of PPARβ. The chondroitin sulphate proteoglycan protein NG2 is one of the most reliable and widely-used markers of OPC (6,30-32), whereas upon differentiation, these OPCs generate oligodendrocytes, which can be stained with myelin marker MBP. In order to assess the differentiation of OPC into oligodendrocytes, first we prepared OPCs from the cerebellar tissue of 1 day old mouse neonates, cultured for 3-4 days and then treated with 1 μM DBB, 0.5 μM DBP and 0.5 μM MePA. After 24 hrs of treatment, OPCs were immunostained with NG2 and MBP. Accordingly, no treatment control group displayed undifferentiated OPCs with strong NG2, but no MBP (FIG. 1M, 1st Panel). However, treatment with all three ligands differentiated OPC into oligodendrocytes as indicated with elevated level of MBP accompanied with strong decline of NG2 (FIG. 1M). Moreover, the formation of oligodendroglia-like branches and quantification of mean fluorescence intensities (MFI) of MBP (FIG. 1N) further corroborated that all three ligands of PPARβ are potent inducers of oligodendroglia differentiation. That result was further confirmed by a Pearson correlation analyses of MFIs (FIG. 1O) between NG2 and MBP, which displayed a strong negative relationship with r=−0.4023 and p<0.001. Similarly, a double-immunofluorescence analysis of another OPC marker A2B5 (FIG. 1P) with myelin protein PLP also revealed a strong downregulation of A2B5 (Supplementary Fig.) with upregulation of PLP. The result was further confirmed with MFI quantification of PLP (FIG. 1Q) and a negative correlation (r=−0.502; p<0.001) between A2B5 and PLP (FIG. 1R).

Next, we wanted to explore if all these endogenous regulators of PPARβ could promote myelination. To test that possibility, OPCs were plated on a randomly aligned polycaprolactone (PCL) nanofiber matrix for 48 hrs. After that, cells were treated with 1 μM DBB, 0.5 μM DBP and 0.5 μM MePA for another 24 hrs and then stained with mature myelin marker MBP. Interestingly, we observed all three compounds strongly stimulated the formation of myelin sheath on these nanofibers as evident with beaded myelin components around the nanofibers indicated by white arrowhead (FIG. 1S). We also quantified number of MBP-ir cells per sq.mm of nanofiber matrix (FIG. 1T) with clear and round cell bodies as indicated with blue arrowheads. Quantitative estimation of these MBP-ir cell bodies indicated that DBB, DBP and MePA stimulated the maturation of oligodendrocytes and hence promoted the synthesis of myelin. In summary, our results suggest that DBB, DBP and MePA stimulated the transcriptional activity of PPARβ, upregulated the expression of myelin genes, stimulate the differentiation of OPCs into oligodendrocytes and promote myelin formation.

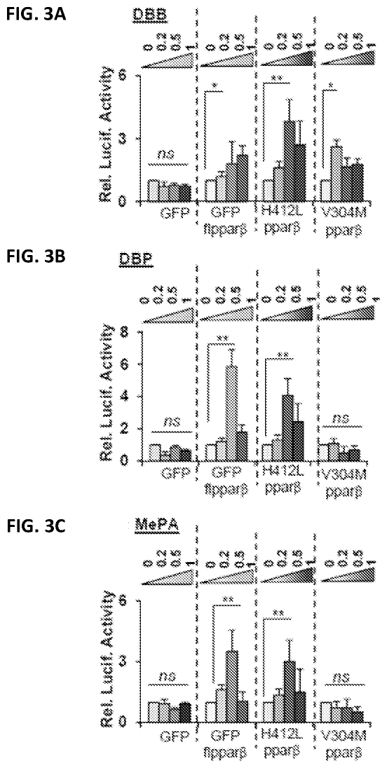

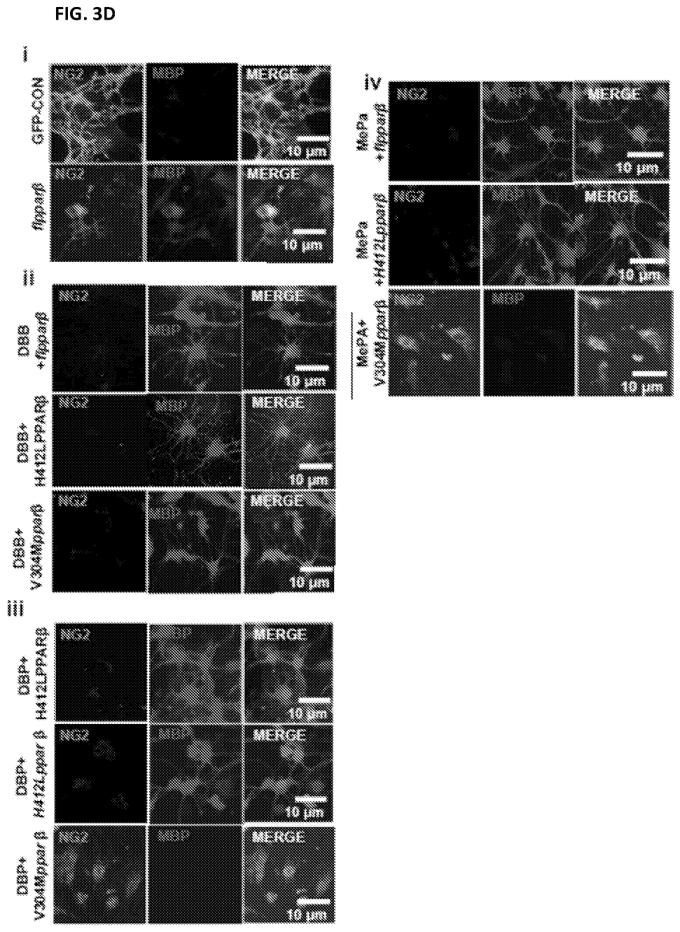

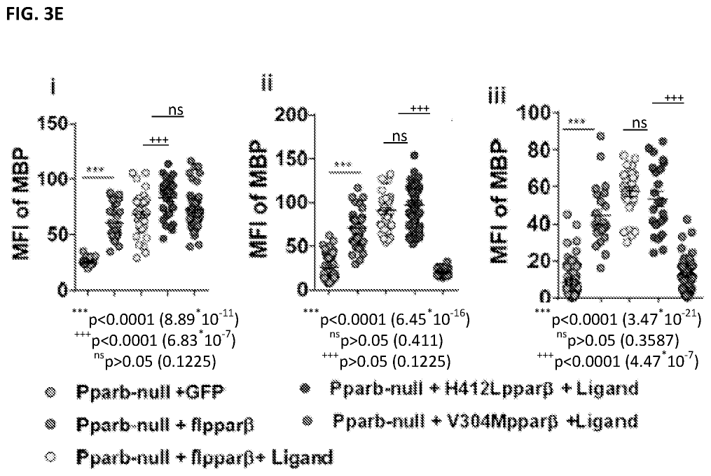

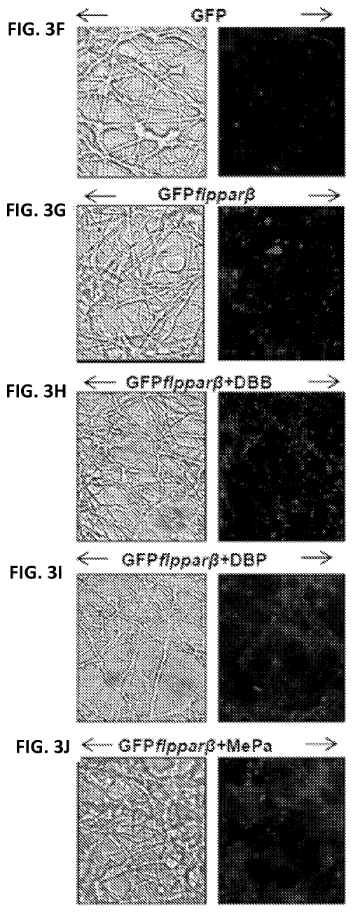

The essential role of PPARβ in DBB-, DBP- and MePa-mediated differentiation of OPCs to oligodendrocytes and myelination. Small molecules often bind to non-specific protein targets and therefore display off-target effects in a biological system. Therefore, there is a possibility that DBB, DBP and MePA might not require PPARβ for the differentiation of oligodendrocytes. To nullify that possibility, we assessed the effect of these compounds on PPRE-luciferase activity in Pparb-null OPCs, reconstructed with either empty vector (FIG. 2A) or pparb gene (FIG. 2B). Briefly, Pparb-null OPCs were transduced with lentiviral particles (MOI: 100) of GFP and GFPflpparβ constructs. After 48 hrs of transduction, cells were transfected with PPRE-luciferase for another 24 hrs and then treated with 1 μM DBB, 0.5 μM DBP and 0.5 μM MePA for additional 5 hrs. After that, PPRE-luciferase activity was assessed. Interestingly, all these compounds are unable to stimulate PPRE luciferase activity in empty vector-constructed Pparb-null OPCs suggesting that PPARβ, but not other PPARs, is required for the ligands-mediated stimulation of PPRE-luciferase activity. Nevertheless, the reconstruction of PPARβ itself significantly restored the PPRE-luciferase activity and that effect was further upregulated with different ligands of PPARβ reiterating that all these ligands signals only through PPARβ. To further confirm the essential role of PPARβ, next we compared PPRE-luciferase activity in different groups of Pparb-null OPCs infected with empty vector, flpparβ, V304Mpparβ and H412Lpparβ constructs of PPARβ. Accordingly, consistent to our GCMS analyses described above, increasing doses of DBB increased PPRE-luciferase activity (FIG. 3A) equivalently in both GFPflpparβ- (FIG. 3A) and GFPV304Mpparβ (FIG. 3A) -infected Pparb-null OPCs. Interestingly, DBB treatment increased PPRE-luciferase activity in GFPH412Lpparβ-transduced Pparb-null OPCs (FIG. 3A) significantly higher than flpparβ group indicating that H412Lpparβ is a ligand-active mutant of PPARβ. Similarly, different doses of both DBP (FIG. 3B) and MePA (FIG. 3C) increased PPRE-luciferase activity in both GFPflpparβ- and GFPH412Lpparβ-transduced, but not GFPV304Mpparβ-infected Pparb-null OPCs suggesting that V304M mutant of PPARβ nullifies the binding of partial polar ligands such as DBP and MePA in its LBD. Next, we wanted to study if these ligands required PPARβ for the differentiation of OPCs to oligodendrocytes. To verify, we performed dual immunofluorescence analyses of NG2 and MBP in DBB- (FIG. 3Dii), DBP- (FIG. 3Diii) and MePA- (FIG. 3Div) treated Pparb-null OPCs, transduced with different constructs of PPARβ. We observed that 1 μM DBB strongly upregulated the expression of MBP in flpparβ-transduced Pparb-null OPCs and stimulated the differentiation to oligodendrocytes with well-defined branches (FIG. 3Dii; first panel). Interestingly, that effect was unaltered in V304Mpparβ- (FIG. 3Dii; second panel) and H412Lpparβ- (FIG. 3Dii; third panel) infected OPCs. In fact, H412L, but not V304M mutant of PPARβ, significantly stimulated the differentiation process as shown in the MFI quantification (FIG. 3Ei). On the other hand, both DBP (FIG. 3Diii) and MePA (FIG. 3Div) increased the expression of MBP in flpparβ- and H412Lpparβ-, but not V304Mpparβ-infected Pparb-null OPCs suggesting that both DBP and MePA required interaction with V304 residue of PPARβ for the differentiation of OPCs to oligodendrocytes. The result was further confirmed by quantifying MFI of MBP in differentiated oligodendrocytes after treatment with DBP (FIG. 3Eii) and MePA (FIG. 3Eiii). Next, we wanted to study if these ligands employ PPARβ for the synthesis of myelin. That possibility was tested with the reconstruction of flpparβ in Pparb-null OPCs seeded in a nanofiber matrix followed by the treatment with 1 uM of DBB (FIG. 3H)), 0.5 μM of DBP (FIG. 3I) and MePA (FIG. 3J). The effect of ligands was compared with Pparb-null OPCs infected with empty vector (FIG. 3F) and flpparβ (FIG. 3G) alone. Interestingly, re-establishment of pparβ gene alone restored the synthesis myelin wraps around nanofibers. That effect was further stimulated with the treatment of ligands suggesting the necessary and sufficient role of PPARβ for the DBB-, DBP- and MePA-stimulated differentiation and maturation of oligodendrocytes. Taken together, our results suggest that DBB, DBP and MePA require PPARβ for the differentiation of OPCs into oligodendrocytes and myelinogenesis. Moreover, our data clearly indicate that all three ligands require interactions with V304 and H412L at PPARβ LBD to achieve these biological activities.

### DISCUSSION

Since its discovery in early 90's (33), the role of PPARβ is widely acknowledged in suppressing inflammation, enhancing oligodendroglial biogenesis and myelination. However, until now there is no physiological regulator known for this receptor. The identification of biological ligands of PPARβ has been described herein. By way of non-limiting example, DBB, DBP and MePA have been shown to be ligands of PPARβ in the mouse brain cerebellum. A series of rigorous experimental procedures confirmed the specificity DBB, DBP and MePA towards PPARβ. First, with the help of EI-GCMS analysis, ionic spectra were detected for DBB, DBP and MePA in PPARβ-, but not PPARα-pulled down cerebellar lysates, which suggests that these ligands are selective ligands of PPARβ. Second, in silico-based high-throughput strategy was performed to screen 22 structurally related aromatic compounds of DBB and DBP. These compounds are categorized based on the type of substituents such as two tertiary butyl groups (n=6), one tertiary butyl group (n=5) and relatively shorter alkyl groups (n=10). Combining thermodynamic binding parameters, bulkiness of the substituents and thermal profile of the protein-ligand complex, these 22 molecules were graded. Based on that evaluation, we have found that di-tert-butyl substituted aromatic compounds including DBB and DBP are the strongest ligands of PPARβ. Third, protein thermal shift assays of PPARβ were performed with increasing concentrations of these compounds. Accordingly, we observed that DBB, DBP and MePA elicited a very large shift in the melting curve of PPARβ with ΔTₘ of more than 2° C. However, these compounds did not show any shift in the melting curve of PPARα protein suggesting that DBB, DBP and MePA are selective agonists of PPARβ, but not PPARα. Finally, TR-FRET analyses of PPARβ LBD with different concentrations of DBB, DBP and MePA resulted in characteristic sigmoid binding curves. Accordingly, the resultant EC₅₀ values (<1 μM) of TR-FRET analyses suggest that all three ligands display high affinity towards PPARβ LBD.

While analyzing the interaction of PPARβ with all three ligands in a molecular level, we observed that these compounds exhibited high affinity to a hydrophobic motif of LBD that contains three conserved non-polar residues namely, V297, L302 and V304. These three non-polar residues create a high affinity interaction interface that can engage DBB, DBP and MePA through hydrophobic interactions. However, due the large alkyl side chains of 16 carbons, MePA displayed a widespread distribution in the ligand binding pocket of PPARβ. In one end, aliphatic sidechain of MePA makes hydrophobic interaction with V297, L302 and V304 located at S2 and S3, two anti-parallel β-strands of PPARβ LBD, whereas on the other hand, the polar ester group of MePA forms an electrostatic interaction with H412 residue of histidine-rich H11 α-helix. Accordingly, our cheminformatic analysis predicted a strong H-bond interaction between H412 and carboxyl head of MePA at a distance of 2.113 Å. Our in silico modeling data is consistent with previous crystallographic study, in which an x-ray crystallographic analysis (28) of PPARβ complexed with pharmacological agonist GW501516 has revealed that H413, a human equivalent of H412, is involved in H-bond interaction with the acidic side chain of the ligand. On the other hand, V298, L303 and V305 located at two anti-parallel beta-strands stabilize the hydrophobic side chain of GW501516. These three hydrophobic amino acids are human equivalent of V297, L302 and V305 of mouse PPARβ respectively. However, among these three amino acids, V304 was found to be positioned adjacent to the tertiary butyl group of DBB, DBP and aliphatic sidechain of MePA at a distance of less than 3 Å. In order to confirm the hydrophobic interaction, V304 residue was mutated to Methionine (V304M). Although, both isopropyl sidechain of valine and thiomethylbutyl sidechain of methionine are equally nonpolar, the sulfur atom of thioether group in methionine sidechain has a lone-pair electron that partially disrupts the VW interaction and therefore, it was expected that V304M mutation would disturb the hydrophobicity of S3 β-strand and might push these ligands away from their contact surface. Interestingly, that V304M mutation indeed abrogated PPARβ's interaction with DBB, DBP and MePA as confirmed with GCMS analysis in Pparb-null OPCs transduced with V304MPPARβ. Moreover, a PPRE-luciferase assay further confirmed that these ligands were unable to stimulate the transcriptional activity of PPARβ in Pparb-null OPCs infected with V304MPPARβ. On the other hand, to confirm the role of H412 in forming the H-bond interaction with MePA, we mutated H412 to a nonpolar Leucine (H412L). Surprisingly, H412L mutation significantly enhanced the interaction of PPARβ with all three ligands as confirmed with EI-GCMS analyses and PPRE-luciferase assay in Pparb-null OPCs transduced with H412L PPARβ. The enhancement of protein-ligand interactions of DBB, DBP and MePA with H412LPPARβ might be due to the augmentation of additional hydrophobicity in the LBD.

Although the biosynthesis of DBB, DBP remains unclear in vertebrates, synthesis of phenolic compounds including phenolic acids, flavonoids, stilbenes and phenylethanoids are well established in plant and prokaryotic organisms. Therefore, regular consumption of vegetables and the distribution of microbiota in gastrointestinal (GI) system might supplement the metabolic needs of these compounds in vertebrate. However, with the help of GCMS, we were able to detect DBB, DBP and MePA in purified OPCs, a cell type which is metabolically active since early embryogenesis, much before the development of GI system. Therefore, the bioavailability of these molecules is an outcome of complex metabolic events that yet to be characterized.

OPCs are highly proliferative and migratory pluripotent cells that rapidly differentiate to oligodendrocytes in the white matter of CNS during development, adulthood and different demyelinating diseases. Therefore, once ligands of PPARβ were identified in OPCs, we next investigated if these compounds stimulated the differentiation of OPCs into oligodendrocytes. Interestingly, DBB, DBP and MePA upregulated the expressions of different myelin markers such as MBP, PLP, MOG and CNPase in purified OPCs. As a molecular mechanism, PPARβ was previously shown (19) to be directly recruited in the promoters and stimulated the transcriptions of these myelin genes. While analyzing the morphological changes, we have observed that upon treatment with all these ligands, OPCs lost their round-shaped morphology and acquired oligodendroglial architecture with highly branched cell processes. Moreover, that differentiation process was accompanied with the loss of OPC markers such as NG2 and A2B5 and upregulation of myelin markers including MBP and PLP. Interestingly, these ligands were unable to transform Pparb-null OPCs into oligodendrocytes, whereas lentiviral reconstruction of flpparb gene was sufficient to restore that differentiation process. The maturation process is further enhanced with the treatment of the ligands suggesting that PPARβ is essential for the ligand-induced differentiation and maturation of oligodendrocytes. A detailed molecular study further confirmed that the interaction of these ligands with V304 and H412 residues of PPARβ LBD had been critical for oligodendroglial differentiation process. V304M mutation of PPARβ completely nullified DBP- and MePA-induced differentiation of OPCs to oligodendrocytes. On the other hand, H412L mutation did not inhibit, but stimulated that differentiation process.

Synthetic agonists of PPARβ had been tested before in a different preclinical model. GW501516, a potent agonist of PPARβ, promoted the development of breast cancer (34), whereas administration of another ligand GW0742 stimulated intestinal cancer (35). Therefore identifying physiological ligands of PPARβ with lesser side-effects is of great interest in battling demyelinating disorder like multiple sclerosis. DBB, DBP and MePA are three such unique molecules, which are biologically available in cerebellum and effectively promote the synthesis of myelin. The effect of these molecules on the synthesis of myelin wrap was tested in OPCs seeded on a nanofiber matrix. All these molecules were observed to promote the formation of myelin wraps around the nanofibers as early as 24 hrs. Studies will be conducted to determine the effects of these molecules on promoting myelination in vivo in different mouse models of demyelinating diseases.

Materials and Methods:

Animals and Reagents: Six to eight weeks old Pparb-null, Ppara-null, and wild-type animals were purchased from Jackson and housed in ventilated cages in a light-, temperature- and humidity-controlled vivarium (7:00 A.M./7:00 P.M. light cycle; 21-23° C.; humidity 35-55%). Animals were given standard mouse chow and water ad libitum and closely monitored for health and overall well-being daily by veterinary staff and the investigators. Animals are bred to generate sufficient male mice according to our approved IACUC protocol. Chemicals including 2,4,6-Tri-ter-butyl Phenol (Cat #T49409), 2,4-Di-tert butyl Phenol (Cat #137731), 2,6-Di-tert butyl Phenol (Cat #D48400), 2-tert butyl-6 methyl Phenol (Cat #B97607), butylated hydroxyl toluene (Cat #PHR1117), 2,4-Di-isopropyl Phenol (Cat #557811), 1,3-Di-tertbutyl benzene (Cat #272051 were purchased from Sigma Aldrich. Rabbit polyclonal anti-PPARβ antibody (SCBT; Cat #sc74517, dilution 1:500; WB and IHC), mouse anti-MBP antibody (SCBT, Cat #sc271524 for WB dilution 1:500; Millipore, at #MAB386 for IF, dilution 1:100), rabbit polyclonal anti-NG2 antibody (Millipore; Cat #AB5320; IF), mouse anti-A2B5 antibody (Millipore; Cat #MAB312; IF), rabbit anti-PLP antibody (Abcam; Cat #ab28486; WB at a dilution of 1:1000 and IF 1:200 dilution), Rabbit anti-MOG antibody (Abcam; Cat #ab109746; WB at a dilution of 1:1000 and IF 1:200 dilution) were used in this study.

Lentiviral Cloning of Flpparb, V304Mpparb and H412Lpparb:

Mouse PPARβ ORF cloned in pLenti-C-mGFP-P2A-Puro vector (cat #MR207001L4) was purchased from Origene. That plasmid was mutated at H412 and V304 residues with leucine and methionine respectively with the help of site-directed mutagenesis strategy. Site-directed mutation was performed with a kit available in Stratagene, as described before (36). After mutation, mutated plasmids were cloned and amplified in Escherichia coli (DH5-a strain) competent cells. Clones were sequenced at ACGT Inc followed by packaging in 293FT cells as described before (36). After 48 hrs of packaging in 293FT cells, viral particles were collected from the sup, concentrated with lenti-concentrator solution (clonetech) and MOI was calculated.

Gas Chromatography Mass Spectra Analysis of PPARβ-Ligand Interaction

Sample preparation: After treatment, mouse primary OPCs were homogenized in ice-cold nondetergent hypotonic buffer [10 mM HEPES (pH 7.9), 1.5 mM MgCl₂, 10 mM KCl, 100 mM DTT, protease and phosphatase inhibitor cocktail]. After 10 min of additional incubation in the hypotonic buffer, the homogenate was centrifuged at 8,000 g at 4° C. for 10 min. Next, the pellet was homogenized in ice-cold extraction buffer [10 mM HEPES (pH 7.9), 1.5 mM MgCl₂, 0.21 M NaCl, 0.2 mM EDTA, 25% (v/v) glycerol, 100 mM DTT, protease and phosphatase inhibitor cocktail], placed on a rotating shaker at 4° C. for 1 h, and then centrifuged at 18,000 g for 10 min. The supernatant (nuclear fraction) was incubated with 1.5 μg of GFP-PPARβLBD at 4° C. for 6 h in a rotating shaker. The reaction mixture was passed through GFP affinity column (Vector Fusion-Aid GFP Kit; Cat #MB-073)), washed four times [50 mM Tris HCl (pH 7.4), 100 mM NaCl, protease and phosphatase inhibitor cocktail] and then eluted with 5M NaCl. The eluted fraction was then transferred to methanol: chloroform: water (4:3:1) mixture and then centrifuged at 14,000 rpm for 90 sec. The organic phase was collected, evaporated in the speedvac, reconstituted with 30 μL chloroform, and finally analyzed with GCMS. In another case, Pparbeta-null OPCs were transduced with lentiviral particles conjugated with GFP-conjugated H412L and V304M PPARβ followed by purification with GFP-column. After that, the eluate was collected from the column with 5M NaCl solution, concentrated with PD-10 desalting column and analyzed for GCMS.

GCMS analyses: Ligand detection: A JEOL GCMate II (JEOL USA, Peabody Mass.) mass spectrometer was used in ligand detection experiments. The gas chromatograph (GC) was an Agilent 6890Plus (Wilmington Del.) equipped with a G1513A auto-injector with 100 vial sample tray connected to a G1512A controller. The gas chromatography column was a fused silica capillary column with a nonpolar 5% phenyl 95% dimethylpolysiloxane phase (Agilent HP-5 ms), 30 meters long, 0.25 mm internal diameter, 0.25 μm film thickness. The carrier gas was Helium (99.9995% Research Grade) run through a STG triple filter (Restek Corp.) at a constant flow rate 1.1 mL/min. The injector was held at 275° C. and was fitted with an Agilent 4 mm ID single taper split liner containing deactivated glass wool. One μL of solution was injected at a split ratio of 20:1. The initial oven temperature was 40° C. held at 2 min, raised to 300° C. at a rate of 10° C. Total run time was 45 min. Data analysis was performed using the TSS Pro software (Shrader analytical & Consulting Laboratories, Inc., Detroit Mich.) provided with the spectrometer. Reconstructed ion current (RIC) chromatographic peaks using ions unique to each compound were used for quantitation. Mass calibration was performed using perfluorokerosene (PFK).

Ligand validation: ISQ7000, a single quadrupole mass spectrometer (Thermofisher Scientific) was used in ligand validation experiment. The GC was a TRACE1310 equipped with a manual injector and a TG-SQC GC column, which is 30 meter long, 0.25 mm wide and 0.25 μm thick. The carrier gas was 99.999% helium. During experiment the oven temperature was initiated at 40° C. for 1 min, ramped up to 250° C. at a rate of 30° C./min and held at 250° C. for 10 min. Total run time was 18 min. Data analysis was performed at Chromeleon 7 chromatography studio.

In Silico structural analyses of PPARβ complexed with DBB, DBP and MePA: Chemical structures of DBB, DBP, MePA, and different other phenolic compounds were derived from ZINC AC ligand docking database. The crystal structure of mouse PPARβ protein is unknown. Therefore we performed homology modeling analyses in Swiss Model online tool considering 3GWX.pdb as a template. Ligands were docked with the help of Swiss Dock docking tool, which generated 100 docked structures as a function of free energy change (ΔG). More is the negative ΔG, stronger is the interaction. For ligand strain energy, we specified a 5å region of the receptor from the centroid of the ligand to be flexible so that the protein structure was relaxed in the computation of the binding energy of the ligands.

TR-FRET analysis: TR-FRET assay was performed using Lanthascreen TR-FRET PPAR-β coactivator assay kit (Cat #PV4685). In this assay, different doses of DBB, DBP and MePA were incubated with GST-tagged recombinant PPARα LBD protein, Terbium (Tb)-tagged anti GST antibody and Fluorescein (FL)-tagged PGC-1α as directed in the manufacturer's protocol. The entire reaction was set up in corning 96 well-plate followed by incubation in a dark place for 30 mins, and then analyzed “Victor X5” machine equipped with dichroic mirror. The excitation wavelength and emission wavelength were set at 340 nm and 540 nm wavelength respectively.

Thermal shift assay: Thermal shift assay was performed in ABI 7500 and QuantStudio 3 real-time PCR machines with commercially available thermal shift dye kit (Life technologies; Cat #4461146). For each reaction, purified protein (0.5 μg to 1 μg) was added to 18 μL of thermal shift buffer provided with the kit, and 1-2 μL of dye. Reaction was set 96 well PCR plate in a dark condition and then placed in the thermal cycler machine using the protocol described elsewhere (25).

Cell culture analysis of OPC: Brain cerebellar tissue was dissected out from P1 pups and dissociated thoroughly in HBSS. Cells were suspended in OPC medium [Dulbecco's modified Eagle's media with L-glutamine, 4.5 g/L glucose and sodium pyruvate (DMEM, Gibco), supplemented with 10% Fetal Bovine Serum (FBS: BioWhittaker; Lonza) and a 1% Antibiotic Antimitotic Solution (Sigma)] and centrifuged at 1000 rpm for 10 minutes. Pellet were resuspended in 10 ml of OPC medium and seeded in 75 cm2 Poly-L-Ornithine coated flasks in a final volume of 12 ml per flask for 12 more days. During that period flasks were kept at 37° C. and 5% CO2. After one day of seeding, the OPC medium was replaced with OPC medium supplemented with PDGF-AA (1 Ong/ml, Millipore). To purify OPCs, the culture flasks were removed from the incubator (after 10-15 days), and the flasks were shaken overnight (18-20 h) in an orbital shaker at 250 rpm and 37° C. OPCs were collected rapidly and passed through a 40 μm nylon mesh strainer placed over a 50 ml conical tube. The flow-through was collected and centrifuged at 1000 rpm, for 10 minutes, and the pellet was then resuspended in 10 ml of OPC medium. These cells were plated on an untreated plastic Petri dish at 37° C. and left for 45 minutes, allowing the microglial cells to attach but not the OPCs. The unattached cells were collected as purified OPCs.

Semi-quantitative RT-PCR and Real-time PCR Analysis: Total RNA was digested with DNase and RT-PCR was carried out as described earlier (37,38) using a RT-PCR kit from Clontech. GAPDH (glyceraldehyde-3-phosphate dehydrogenase) was used to ascertain that an equivalent amount of cDNA was synthesized from different samples. Quantitative RT PCR was performed in the ABI-Prism 7500 standard qPCR system (Applied Biosystems, Foster City, Calif.) as described earlier (37,38) using TaqMan Universal Master mix and FAM-labeled probes and primers (Applied Biosystems). Data were processed by the ABI Sequence Detection System 1.6 software and analyzed by ANOVA.

Immunoblot analysis: For whole-cell and tissue lysates, samples were homogenized in RIPA buffer+protease and phosphatase inhibitors (Sigma), passed through a 26-gauge needle, rotated end over end for 30 min at 4° C., and centrifuged for 10 min at 18,000×g. Protein concentrations were determined using a NanoDrop 2000 (Thermo Fisher), and 15-30 μg sample was heat-denatured and resolved on 10% or 12% polyacrylamide-SDS gels, transferred to 0.45 μm nitrocellulose membranes under semidry conditions (15V for 12 min). Membranes were blocked for 1 h with blocking buffer (Li-Cor), incubated with primary antibodies overnight at 4° C. under shaking conditions, washed, incubated with IR-dye-labeled secondary antibodies (1:17,000; Li-Cor) for 45 min at room temperature, washed, and visualized with the Odyssey Infrared Imaging System (Li-Cor). Blots were converted to grayscale and then binary, analyzed using Fiji, and normalized to appropriate loading controls.

Electron Microscopy and analysis: Six-to-eight weeks old C57BL/6 and PPARβ (−/−) mice were perfused with 2% glutaraldehyde and 2% paraformaldehyde followed by storage of their brains at 4% paraformaldehyde for 48 hours. Brains were sectioned in sagittal plane with 50-60 μm thickness, processed with series of buffers including sodium cacodylate buffer, osmium tetroxide and uranyl acetate before embedding in a block. After that, 500-600 nm sections were cut and stained with toluidine blue to verify tissue integrity and thickness. Ultrathin sections with corpus callosum (CC) were cut at a thickness of 70 nm and then imaged in electron microscopy at University of Illinois, Chicago core facility. G ratio, an index of myelin thickness, was measured with the ratio of inner versus outer radius of myelin membrane (n=5 per group). Approximately, 130-220 randomly-selected myelin fibers were selected for g-ratio calculations and plotted as a scattered plot in Graphpad prism software. Statistical significance was tested using the unpaired t-test for two different groups and one-way ANOVA with treatment as independent variables for more than two groups. While comparing between wild-type and PPARβ (−/−) animals, we applied two-way ANOVA with both treatment and genotype as independent factors.

Immunohistochemical analysis: Analyses were done as described elsewhere (29,38).

Statistical analyses: All values are expressed as the mean±SD. Differences among means were analyzed using one- or two-way ANOVA with time or genotype as the independent factors. Differences in behavioral measures were examined by independent one-way or repeated-measures ANOVAs using SPSS. Homogeneity of variance between test groups was examined using Levene's test. Post-hoc analyses of between-subjects effects were conducted using Scheffe's, Tukey's or Games-Howell tests, where appropriate. p<0.05 was considered statistically significant.

In Vivo Determination of Promotion of Myelination by PPARβ Ligands Using Different Mouse Models of Demyelinating Diseases.

Studies will be conducted to determine the effects of PPARβ ligands on promoting myelination in vivo. By way of non-limiting example, the in vivo models include Experimental autoimmune Encephalomyelitis (EAE) (MS like demyelination), Cuprizone intoxication (Demyelination), GaIC Twicher mice (Krabbe disease like demyelination), ABCD1 knockout mice (X-ALD and AMN like demyelination) and Experimental Autoimmune Neuritis (EAN) (Optic neuritis like demyelination). The PPARβ ligands to be tested will include 1,3-Di-tertbutyl benzene (DBB), 2,4-Di-tertbutyl phenol (DBP), Methyl palmitate (MePA), 2,6-Di-tertbutyl-4-Methyl Phenol (DBMP), 3,4-Di-tertbutyl-Phenol (3,4-DBP), 2,3-Di-tertbutyl-Phenol (2,3-DBP), and 2,6-Di-tertbutyl-Phenol (2,6-DBP).

The above Figures and disclosure are intended to be illustrative and not exhaustive. This description will suggest many variations and alternatives to one of ordinary skill in the art. All such variations and alternatives are intended to be encompassed within the scope of the attached claims. Those familiar with the art may recognize other equivalents to the specific embodiments described herein which equivalents are also intended to be encompassed by the attached claims.

### REFERENCES

1. Doering, L. C., and Fedoroff, S. (1984) Isolation and transplantation of oligodendrocyte precursor cells. J Neurol Sci 63, 183-196.
2. Clarke, L. E., Young, K. M., Hamilton, N. B., Li, H., Richardson, W. D., and Attwell, D. (2012) Properties and fate of oligodendrocyte progenitor cells in the corpus callosum, motor cortex, and piriform cortex of the mouse. J Neurosci 32, 8173-8185.
3. Valerio-Gomes, B., Guimaraes, D. M., Szczupak, D., and Lent, R. (2018) The Absolute Number of Oligodendrocytes in the Adult Mouse Brain. Front Neuroanat 12, 90.
4. Sgaier, S. K., Millet, S., Villanueva, M. P., Berenshteyn, F., Song, C., and Joyner, A. L. (2005) Morphogenetic and cellular movements that shape the mouse cerebellum; insights from genetic fate mapping. Neuron 45, 27-40.
5. Hashimoto, R., Hori, K., Owa, T., Miyashita, S., Dewa, K., Masuyama, N., Sakai, K., Hayase, Y., Seto, Y., Inoue, Y. U., Inoue, T., Ichinohe, N., Kawaguchi, Y., Akiyama, H., Koizumi, S., and Hoshino, M. (2016) Origins of oligodendrocytes in the cerebellum, whose development is controlled by the transcription factor, Sox9. Mech Dev 140, 25-40.
6. Nishiyama, A., Komitova, M., Suzuki, R., and Zhu, X. (2009) Polydendrocytes (NG2 cells): multifunctional cells with lineage plasticity. Nat Rev Neurosci 10, 9-22.
7. Buffo, A., and Rossi, F. (2013) Origin, lineage and function of cerebellar glia. Prog Neurobiol 109, 42-63.
8. Allerand, C. D., and Murray, M. R. (1968) Myelin formation in vitro. Endogeneous influences on cultures of newborn mouse cerebellum. Arch Neurol 19, 292-301.
9. Allerand, C. D. (1971) Patterns of neuronal differentiation in developing cultures of neonatal mouse cerebellum: a living and silver impregnation study. J Comp Neurol 142, 167-203.
10. Kucharova, K., and Stallcup, W. B. (2010) The NG2 proteoglycan promotes oligodendrocyte progenitor proliferation and developmental myelination. Neuroscience 166, 185-194.
11. Issemann, I., and Green, S. (1990) Activation of a member of the steroid hormone receptor superfamily by peroxisome proliferators. Nature 347, 645-650.
12. Dreyer, C., Krey, G., Keller, H., Givel, F., Helftenbein, G., and Wahli, W. (1992) Control of the peroxisomal beta-oxidation pathway by a novel family of nuclear hormone receptors. Cell 68, 879-887.
13. Desvergne, B., and Wahli, W. (1999) Peroxisome proliferator-activated receptors: nuclear control of metabolism. Endocr Rev 20, 649-688.
14. Woo, C. H., Massett, M. P., Shishido, T., Itoh, S., Ding, B., McClain, C., Che, W., Vulapalli, S. R., Yan, C., and Abe, J. (2006) ERK5 activation inhibits inflammatory responses via peroxisome proliferator-activated receptor delta (PPARdelta) stimulation. J Biol Chem 281, 32164-32174.
15. Jana, M., and Pahan, K. (2012) Gemfibrozil, a lipid lowering drug, inhibits the activation of primary human microglia via peroxisome proliferator-activated receptor beta. Neurochem Res 37, 1718-1729.
16. Dunn, S. E., Bhat, R., Straus, D. S., Sobel, R. A., Axtell, R., Johnson, A., Nguyen, K., Mukundan, L., Moshkova, M., Dugas, J. C., Chawla, A., and Steinman, L. (2010) Peroxisome proliferator-activated receptor delta limits the expansion of pathogenic Th cells during central nervous system autoimmunity. J Exp Med 207, 1599-1608.
17. de la Monte, S. M., and Wands, J. R. (2006) Molecular indices of oxidative stress and mitochondrial dysfunction occur early and often progress with severity of Alzheimer's disease. J Alzheimers Dis 9, 167-181.
18. Jana, M., and Pahan, K. (2005) Redox regulation of cytokine-mediated inhibition of myelin gene expression in human primary oligodendrocytes. Free Radic Biol Med 39, 823-831.
19. Jana, M., Mondel, S., Gonzalez, F. J., and Pahan, K. (2012) Gemfibrozil, a lipid-lowering drug, increases myelin genes in human oligodendrocytes via peroxisome proliferator-activated receptor-beta. J Biol Chem 287, 34134-34148.
20. Almad, A., and McTigue, D. M. (2010) Chronic expression of PPAR-delta by oligodendrocyte lineage cells in the injured rat spinal cord. J Comp Neurol 518, 785-799.
21. Woods, J. W., Tanen, M., Figueroa, D. J., Biswas, C., Zycband, E., Moller, D. E., Austin, C. P., and Berger, J. P. (2003) Localization of PPARdelta in murine central nervous system: expression in oligodendrocytes and neurons. Brain Res 975, 10-21.
22. Warden, A., Truitt, J., Merriman, M., Ponomareva, O., Jameson, K., Ferguson, L. B., Mayfield, R. D., and Harris, R. A. (2016) Localization of PPAR isotypes in the adult mouse and human brain. Sci Rep 6, 27618.
23. Saluja, I., Granneman, J. G., and Skoff, R. P. (2001) PPAR delta agonists stimulate oligodendrocyte differentiation in tissue culture. Glia 33, 191-204
24. Reilly, S. M., and Lee, C. H. (2008) PPAR delta as a therapeutic target in metabolic disease. FEBS Lett 582, 26-31.
25. Roy, A., Kundu, M., Jana, M., Mishra, R. K., Yung, Y., Luan, C. H., Gonzalez, F. J., and Pahan, K. (2016) Identification and characterization of PPARalpha ligands in the hippocampus. Nat Chem Biol 12, 1075-1083.
26. Hostetler, H. A., Kier, A. B., and Schroeder, F. (2006) Very-long-chain and branched-chain fatty acyl-CoAs are high affinity ligands for the peroxisome proliferator-activated receptor alpha (PPARalpha). Biochemistry 45, 7669-7681.
27. Pawlak, M., Lefebvre, P., and Staels, B. (2015) Molecular mechanism of PPARalpha action and its impact on lipid metabolism, inflammation and fibrosis in non-alcoholic fatty liver disease. J Hepatol 62, 720-733.
28. Wu, C. C., Baiga, T. J., Downes, M., La Clair, J. J., Atkins, A. R., Richard, S. B., Fan, W., Stockley-Noel, T. A., Bowman, M. E., Noel, J. P., and Evans, R. M. (2017) Structural basis for specific ligation of the peroxisome proliferator-activated receptor delta. Proc Natl Acad Sci USA 114, E2563-E2570.
29. Roy, A., Jana, M., Kundu, M., Corbett, G. T., Rangaswamy, S. B., Mishra, R. K., Luan, C. H., Gonzalez, F. J., and Pahan, K. HMG-CoA Reductase Inhibitors Bind to PPARalpha to Upregulate Neurotrophin Expression in the Brain and Improve Memory in Mice. Cell Metab 22, 253-265.
30. Keirstead, H. S., Levine, J. M., and Blakemore, W. F. (1998) Response of the oligodendrocyte progenitor cell population (defined by NG2 labelling) to demyelination of the adult spinal cord. Glia 22, 161-170.
31. Dawson, M. R., Levine, J. M., and Reynolds, R. (2000) NG2-expressing cells in the central nervous system: are they oligodendroglial progenitors? J Neurosci Res 61, 471-479.
32. Stallcup, W. B. (2002) The NG2 proteoglycan: past insights and future prospects. J Neurocytol 31, 423-435.
33. Krey, G., Keller, H., Mahfoudi, A., Medin, J., Ozato, K., Dreyer, C., and Wahli, W. (1993) Xenopus peroxisome proliferator activated receptors: genomic organization, response element recognition, heterodimer formation with retinoid X receptor and activation by fatty acids. J Steroid Biochem Mol Biol 47, 65-73.
34. Wang, X., Wang, G., Shi, Y., Sun, L., Gorczynski, R., Li, Y. J., Xu, Z., and Spaner, D. E. (2016) PPAR-delta promotes survival of breast cancer cells in harsh metabolic conditions. Oncogenesis 5, e232.
35. Gupta, R. A., Wang, D., Katkuri, S., Wang, H., Dey, S. K., and DuBois, R. N. (2004) Activation of nuclear hormone receptor peroxisome proliferator-activated receptor-delta accelerates intestinal adenoma growth. Nat Med 10, 245-247.
36. Roy, A., Jana, M., Corbett, G. T., Ramaswamy, S., Kordower, J. H., Gonzalez, F. J., and Pahan, K. Regulation of cyclic AMP response element binding and hippocampal plasticity-related genes by peroxisome proliferator-activated receptor alpha. Cell Rep 4, 724-737.
37. Roy, A., Fung, Y. K., Liu, X., and Pahan, K. (2006) Up-regulation of microglial CD11b expression by nitric oxide. J Biol Chem 281, 14971-14980.
38. Ghosh, A., Roy, A., Liu, X., Kordower, J. H., Mufson, E. J., Hartley, D. M., Ghosh, S., Mosley, R. L., Gendelman, H. E., and Pahan, K. (2007) Selective inhibition of NF-kappaB activation prevents dopaminergic neuronal loss in a mouse model of Parkinson's disease. Proc Natl Acad Sci USA 104, 18754-18759.

1. A method of modulating peroxisome proliferator-activated receptor (PPARβ) activity in a cell in a subject in need thereof, the method comprising:
administering an effective amount of a PPARβ ligand to the subject, the PPARβ ligand selected from the group consisting of 1,3-Di-tertbutyl benzene (DBB), 2,4-Di-tertbutyl phenol (DBP), Methyl palmitate (MePA), 2,6-Di-tertbutyl-4-Methyl Phenol (DBMP), 3,4-Di-tertbutyl-Phenol (3,4-DBP), 2,3-Di-tertbutyl-Phenol (2,3-DBP), and 2,6-Di-tertbutyl-Phenol (2,6-DBP).

2. The method according to claim 1 wherein the PPARβ ligand is DBB.

3. The method according to claim 1, wherein the PPARβ ligand is DBP.

4. The method according to claim 1, wherein the PPARβ ligand is MePA.

5. The method according to claim 1, wherein the PPARβ ligand is 2,6-Di-tertbutyl-4-Methyl Phenol (DBMP).

6. The method according to claim 1, wherein the PPARβ ligand is 3,4-Di-tertbutyl-Phenol (3,4-DBP).

7. The method according to claim 1, wherein the PPARβ ligand is 2,3-Di-tertbutyl-Phenol (2,3-DBP).

8. The method according to claim 1, wherein the PPARβ ligand is 2,6-Di-tertbutyl-Phenol (2,6-DBP).

9. The method according to claim 1, comprising modulating the PPARβ activity in a central nervous system (CNS) cell.

10. The method according to claim 1, comprising modulating the PPARβ activity in an oligodendroglial progenitor cell (OPC).

11. The method according to claim 1, comprising administering the effective amount of the PPARβ ligand to a subject having a demyelinating disorder.

12. The method according to claim 10, wherein the demyelinting disorder is selected from the group consisting of multiple sclerosis (MS), X-Adrenoleukodystrophy (X-ALD), Adrenomyeloneuropathy (AMN), Neuromyelitis optica (Devic's disease), acute-disseminated encephalomyelitis (ADEM), acute haemorrhagic leucoencephalitis (AHL), progressive multifocal leukoencephalopathy (PML), Krabbe disease and HIV dementia.

13. The method according to claim 1, wherein the effective amount of the PPARβ ligand is delivered by nebulization.

14. The method according to claim 1, wherein the effective amount of the PPARβ ligand is delivered orally.

15. The method according to claim 1, wherein the effective amount of the PPARβ ligand promotes myelination in the CNS.

16. The method according to claim 1, wherein the effective amount of the PPARβ ligand increases differentiation of OPC to oligodendrocytes.
